(19) United States
(12) Patent Application Publication
(10) Pub. No.: US 2019/0199715 A1
(43) Pub. Date: Jun. 27, 2019

(54) SHARED ACCOUNT ENROLLMENT AND BIOMETRIC ACTIVITY MONITORING FOR VALUE APPORTIONMENT

(71) Applicant: PAYPAL, INC., San Jose, CA (US)

(72) Inventor: Ryan Christopher May, Toronto (CA)

(21) Appl. No.: 15/854,616

(22) Filed: Dec. 26, 2017

(51) Int. Cl. H04L 29/06; G16H 10/60

(52) U.S. Cl. CPC H04L 63/0861; G16H 10/60; H04L 63/083

(57) ABSTRACT

There are provided systems and methods for shared account enrollment and biometric activity monitoring for value apportionment. A shared account may be generated to track biometric activity for multiple users and provide a shared value that may be apportioned among the users based on the biometric activity. The account may be set with rewards or goals based on the biometric activity. A service provider may provide a biometric tracking platform having a portal where each user may enter an account code or identifier for the account, and link the account to a biometric tracking device and/or online service that interacts with the biometric tracking device to determine activity data for each user. Based on the activity data, each user may receive portions of the shared value at particular time interfaces for accomplishing the goals. The activity may be tracked in real-time and output through comparison interfaces.

### TECHNICAL FIELD

The present application generally relates to biometric data tracking for multiple users and comparison of user activity and more specifically to providing an online platform that tracks biometric activity data over a period of time to apportion shared value between the users.

### BACKGROUND

A computing device may have various physical sensors that detect real-world conditions proximate to the device and associated with the device, including biometric data for a user and activity data for that user. This may include a mobile communication device (e.g., a smart phone), as well as a wearable computing device (e.g., fitness tracker). The user may utilize the device to track the user's health, activity, and fitness or exercise over time. Additionally, the user may set self-goals to increase their health or fitness. However, unless the user is willing or able to share the biometric data and/or health goals, such information may be limited to only the user, which may limit motivation means available to the user.

### BRIEF DESCRIPTION OF THE DRAWINGS

FIG. 1 is a block diagram of a networked system suitable for implementing the processes described herein, according to an embodiment;

FIG. 2A is an exemplary interaction flowchart for registration biometric devices in a shared value account and tracking of biometric activity data for value apportionment, according to an embodiment;

FIG. 2B is an exemplary application interface to register and enroll in a shared account for biometric activity data, according to an embodiment;

FIG. 3 is an exemplary system environment where a biometric tracking portal may interface with a communication device and biometric tracking device to determine value apportionment based on biometric activity data, according to an embodiment;

FIG. 4 is a flowchart of an exemplary process for shared account enrollment and biometric activity monitoring for value apportionment, according to an embodiment; and

FIG. 5 is a block diagram of a computer system suitable for implementing one or more components in FIG. 1, according to an embodiment.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

Provided are methods utilized for shared account enrollment and biometric activity monitoring for value apportionment. Systems suitable for practicing methods of the present disclosure are also provided.

Users tend to be more incentivized to increase their overall health and activity if the user is engaged in physical activity with other users, or is motivated by other users to exercise. Thus, the user may wish to have motivation to continue to increase their activity by having another user monitor their health or provide the user with activity goals. However, users may be unwilling or share such data freely or make particular health data known to other users. Additionally, some users may not share all of their data, and may not be able to share tracked data over time with other users. Thus, current biometric activity sensors and devices may be inefficient or unhelpful at accomplishing their main goal in increasing a user's health and activity.

One or more users may wish to establish a main or shared account that is accessible to a plurality of users, including the user establishing the account, to monitor and track biometric and activity data (referred to herein as biometric activity data or just activity data), as well as establish a shared value for the account that may be apportioned based on biometric activity over time. The account may be provided to generate a group of users and pool value to allow for value apportionment based on biometric activity. In this regard, the user(s) wishing to establish the account may first access an online service provider that may provide one or more shared account signup and biometric tracking platforms to establish the account. The account may be established by providing account details, such as a login, password (or other authentication credential, such as a biometric fingerprint, retinal scan, etc.), and account creation details, such as a group name for the account and group of users. The online service provider may receive other account creation details, including personal or business information for a user or business establishing the account. In various embodiments, the account may be funded initially by the establishing user(s), for example, through a transaction using a payment instrument, including a transfer from a personal account of the establishing user. The user(s) creating the account may also enter user identifiers for one or more other users that are requested to join the group and share in providing biometric activity. Using the identifiers, the service provider platform may transmit communications to the other users that requests signup and enrollment in the account through their respective personal accounts and biometric activity trackers. For example, an email, text message, or application notification may be sent to various users through their respective identifier, where the message includes an executable process and/or link to access a signup and enrollment portal of the biometric tracking platform. The data may be input through one or more interfaces provided by the platform(s) of the service provider, for example, through an account creation portal that leads the user(s) through one or more signup and account creation interfaces. In this regard, a device used to create the account may access the platform through navigation to the online service provider in an application, and may display the interfaces after accessing the portal and requesting account creation.

In order to create the shared account and/or provide value into the share account, as well as signup and enroll in an established account for biometric activity data tracking, each user establishing and/or enrolling in the account may be required to create their own personal account. The accounts may be provided by the service provider, which may provide account services across multiple different and disparate service provider and merchant platforms. A user may be required to provide identification information to establish the account, such as personal information for a user, business or merchant information for an entity, or other types of identification information including a name, address, and/or other information. The user may also be required to provide financial information, including payment card (e.g., credit/debit card) information, bank account information, gift card information, and/or benefits/incentives, which may be used to provide or receive funds. In order to create an account, the user may be required to select an account name and/or provide authentication credentials, such as a password, personal identification number (PIN), answers to security questions, and/or other authentication information. Once an account is created, the account may be accessed through a web browser from a website of the service provider and/or a dedicated application of the service provider, such as a mobile smart phone application. A token may be issued to the device of the user for their respective personal account, where the token may include data (which may be encrypted) allowing the service provider to identify the user and their account and authenticate the user. Additionally, the online payment provider or other service provider may provide digital wallet services, which may offer financial services to send, store, and receive money, process financial instruments, and/or provide transaction histories.

Once the shared account is established, and any personal accounts are created, individual users may enroll in the shared account through a signup process. The signup process may be accessed through a shared account signup and enrollment portal hosted by the online service provider's biometric tracking platform. The signup process may be directly accessed through navigation to an application interface or webpage for the process. In order to identify the shared account, the user may enter a shared account identifier, which may be provided to the user from the establishing user of the shared account. In certain embodiments, such an identifier may correspond to an account or signup code that uniquely identifies the account and authenticates the user to enroll in the account by being shared only with trusted users that the establishing user wishes to join the account (and therefore shares the code with). The signup process may also be accessed through use of a signup message received by a user based on entry of the aforementioned user, device, or account identifier for the user to a signup notification process provided through the shared account. For example, an executable process or link may navigate a user to the process. The signup and enrollment portal may include multiple processes, including a first process to link a personal account to the shared account and enroll in the shared account through linking the personal account. Linking the personal account may be done through providing an identifier for the user, which may be used to identify the user and communicate through the user, such as an account name or identifier for the personal account of the user. The user may also enter another identifier, such as a phone number or email, which may also be used to enroll the user in the shared account. The user may be required to authenticate the account, phone number, email address, or other identifier used for communication with the user through a message sent to the user and corresponding reply action. Once confirmed, the user may be provided initial enrollment in the shared account.

The application or website of the service provider, such as PayPal®, may provide payments and the other transaction processing services, including transfer services to fund value into the shared account as well as receive value from the share account. In other embodiments, other types of service providers, applications, online transaction processors, social networking and/or social posting/microblogging platforms, or other entities may provide the processes described herein to utilize biometric tracking over a time period to apportion and determine value between users. For example, a social networking platform may provide a service to create a shared account that may allow user profiles on the social networking platform to join the shared account either directly (e.g., through searching or finding a page or account for the shared account) and requesting to join or joining through a signup processing, which may require a code or authentication credential. The social network platform may allow funding into the account through user payments or another transaction processing service. Moreover, the aforementioned platforms, applications, or services may also allow linking of a biometric device, as discussed herein.

If the user wishes to complete enrollment in the shared account so that the service provider may track biometric activity of the user and provide apportionment of account value, the user may also be requested to select a device used to detect biometrics and activity of the user, such as a mobile communication device, a wearable communication device including a fitness tracker, or other type of device that may track a user's activity and/or biometrics. For example, a wearable computing device may correspond to a wearable biometric sensor, smart watch, smart eyeglasses, or other type of wearable device. The device may include a biometric and/or activity sensor, such as a heart rate monitor, pedometer, distance tracker, perspiration detector, or other sensor used to capture a user's biometrics and/or activity. The biometric activity tracking device may be associated with an identifier used to identify and communicate with the biometric activity tracking device, for example, to retrieve biometric and activity data for a user using the device and track the data over one or more time periods. For example, the identifier may be a specific unique device identifier for the device. The device may also be identified through a connected device and/or account. For example, the user may establish a biometric tracking account with an application or another online platform that tracks biometric activity through the device. The user may therefore link the shared account to their biometric tracking application or online account. The identifier may be used to identify the biometric activity tracking device and determine the capabilities of the device, including available fitness and biometric tracking sensors and processes. Additionally, the identifier may be utilized for communication between the service provider and the device, or the application/online platform used to track the user's biometric activity. Biometric activity may therefore be tracked by one or more connected computing devices, as well as multiple devices in communication with each other. Biometric activity, or just activity, may correspond to any human body related health, fitness, or exercise related parameter that may be tracked using one or more devices, including weight, steps, exercise (running, jogging, weight lifting, playing a sport, etc.), heart rate, perspiration, sleep, or other factors that may relate to a person's well-being and overall health.

Using the personal account of the establishing user and/or enrolled users in the shared account, value or funds may be added to the shared account, which may create a pool share that may be apportioned between the enrolled users based on biometric activity tracked over time. For example, the value may be provided by each individual user, and may be required to be evenly split (and therefore even amounts provided) between the users so that each user is required to provide an equal portion of the value or pool. The value may also be unevenly split, or may be provided by one or more main or administrative users, such as the user(s) establishing the shared account. The users may agree to the values provided to the pool, and may utilize their respective account, or another payment instrument, to provide the funds, for example, through a transfer of funds between accounts and/or electronic transaction processing of a payment instrument provided by a user.

After creation of the shared account, the establishing user may create goals, rewards, or other preferences and settings that are established for apportionment of the shared account's value based on the biometric activity of the users. Value sharing settings may therefore correspond to biometric or activity goals, reward settings, or measurements that may cause the service provider's biometric tracking portal to automatically apportion the shared accounts value based on individual and/or group biometrics over a period of time. The settings may also be created, set up, and/or agreed upon by the enrolled user through one or more setting establishment interfaces providing application or webpage interface fields for entry and/or approval of setting data. The settings may correspond to a certain amount or goal amount of activity over a period of time, such as a number of steps, minutes/days/weeks of exercise, distance walked/jogged/run, number of exercises, or other amount of time, distance, or other measurement of activity. The goals may be done over a time period, such as an hour, day, week, or month. The goals may also correspond to a biometric or health measurement, such as a daily average heart rate, heart rate while exercising, weight and/or weight loss/gain, amount of sleep, or other biometric measurement captured by a biometric sensor.

Goals may be set for enrolled users individually, or as a group. The group may correspond to the entire enrolled users, or sub-groups within the enrolled users, such as competing users and/or groups of users (e.g., one half of an office versus another or a management group or boss versus employees). A goal therefore may be set to exceed or beat another group, such as by having biometric activity data that exceeds or beats the other user or group, or exceeds a particular threshold before the other group. In the previous example, the employees may be required to individually or collectively beat a management team, or may be required to exceed 10,000 daily steps before the management team. The goals may also be set so that individual or group users are required to increase their biometric health or activity over a previous time period, such as by having more steps or time active/exercising in a subsequent week or a lower heart rate in a subsequent time period. The goals may be ranked or may have different rewards or value sharing settings so that the goals provide different pro rata shares or portions of the shared accounts value based on accomplishing different goals. Additionally, the goals may be percentage based, so that different users or groups of users may be able to compete even if they fall into different ages, heights, weights, or other category. As such, goals for competing teams or sub-groups may differ based on specific goals, physiological data, or other factors. For example, if the management team has an average age of 55 and is generally overweight, while the employee team has an average age of 28 and is generally fit, the goals for the management team can be significantly lower than for the employee team, e.g., 10,000 steps during the week for the employee team and 4,000 steps for the management team.

Once the value and the value sharing settings are established for the account, and each enrolled user has joined and linked their biometric device, the service provider's biometric tracking platform may interface with tracking applications and processed for the biometric activity tracking devices of each enrolled user to track biometrics and activity over time. The time period may be set in the value sharing settings, and the service provider may continuously monitor the data or pull stored data for the biometric activity of each enrolled user at a specific time, such as at the end of the time period and for the time period. Thus, the biometric activity data may be determined in real-time or at certain intervals. The biometric activity data, the service provider's platform may output one or more tracking interfaces that display data to the enrolled user and/or the establishing user corresponding to the biometric activity of each user over time. The display interfaces for biometric activity may include static or real-time output data that displays various quantitative data of the user's biometrics and activity, such as how long a user has exercised, number of calories burned, steps taken, average or current heart rate, or other activity or biometric marker. The biometric activity output data in one or more interfaces may be tracked over a period of time, and show increases and/or decreases in biometrics or activity, as well as percentage increases or decreases. Such activity may be compared to other users and/or groups so that the user may view a comparison output display showing the users performance versus other users or groups, including those that the user is competing against.

A user may be capable of viewing their biometric activity, as well as their biometric activity performance increases and/or decreases. The user may also be able to view other users' biometric activity, as well as increases and/or decreases. When viewing other users, identifying data of the other users may be removed, scrubbed, or redacted to address privacy issues and concerns of other users and allow sharing of biometric data in an open manner without identifying users. Goals and settings for biometric activity over a time period may be displayed, as well as necessary biometrics or activity to accomplish those settings and earn a corresponding reward, share, or portion of the shared account's pool or value. For example, a user may view their current activity or biometric (e.g., 45 minutes of active walking), and determine what is required to accomplish a set goal (e.g., 60 minutes of daily active walking). The output interface may also display other users close to this goal who may accomplish the goal before the user, or if a reward (e.g., a share or portion of the shared accounts value) for the goal is still obtainable and what the reward is. Additionally, earned shares or pro rata portions/shares of a shared value or pool for the shared account may be displayed to the user, where the earned shares may be for the user, the user's group, and/or other users/groups enrolled in the shared account. Remaining value in the pool may also be displayed, as well as requirements to receive that value, such as a goal requirement and the user's amount applied to the goal (which may show the user what left is required to meet that goal).

Thus, the service provider's biometric tracking platform may also automatically determine, apportion, and/or distribute the shared value or pool for the shared account based on the value sharing settings and each user's or group of users' biometric activity over the time period. The service provider may then use this biometric activity to determine any earned rewards, shares, or portions of the shared account's value. The determination may be done automatically at the end of the time period, and may be done to determine any accrued or earned shares or pro rata portions/shared of the shared account's value for the user. Each individual enrolled user may have their respective biometric data calculated individually and for any joined groups. The individual may receive the share individually or as a group at the end of the time period. The portion of the value determined for the user may be a flat reward, such as a reward for beating another user or group or accomplishing a goal, or may be a percentage fluctuating reward based on an amount or percentage increase or decrease of biometric activity. For example, a portion (e.g., 20%) of the pool may be set for a user having a highest percentage increase in activity, which may earn the entire 20%. The 20% portion may also be given to all users based on their percentage increase or decrease, with a largest percentage increase taking a larger percentage of the portion, which may be a flat amount or may be based on a comparison of the percentage increases/decreases.

In various embodiments, the share or pro rata portion of the shared account's value may be transferred to the user earning the share through an electronic transaction processing service provider by the service provider. For example, the service provider may electronically process a transaction to transfer the share to an account of the user earning the share so that an amount corresponding to the share is withdrawn from the shared account and deposited in the user's personal account. The amount may then be available for the user to utilize when processing transactions using the user's personal account. In various embodiments, the amount provided to the user's personal account for the share may also be limited based on value sharing settings for the account. For example, the amount may be restricted to purchases and/or transactions with only specific merchants and/or for specific items. The shared account may establish a setting where the share's amount may only be used on fitness or health related merchants or items, and may designate the specific items or merchants in certain embodiments. Thus, the user may only use the amount for specific purchases and/or with specific merchants. When the user utilizes the user's personal account with non-qualifying purchases, the amount may not be used from the account; however, with qualifying purchases, the amount may be automatically applied prior to any funds of the user.

Thus, the service provider may provide an online platform and service that allows users to link biometric activity data and track data over time to allow for output of comparison data to users into one or more interfaces. The interfaces may allow for viewing of tracked data between disparate devices without requiring direct interfacing and/or retrieval of device data by each user's individual device, allowing for an online platform to provide and host data and perform data determination and output without utilizing the computing resources of an individual user's device. The service provider's platform may further provide for value apportionment in a shared account automatically based on settings of the account without requiring user input. This processing may be done based on input biometric activity data, and may provide motivation for users to utilize their biometric activity trackers without compromising a user's privacy and/or security by sharing sensitive data with other users.

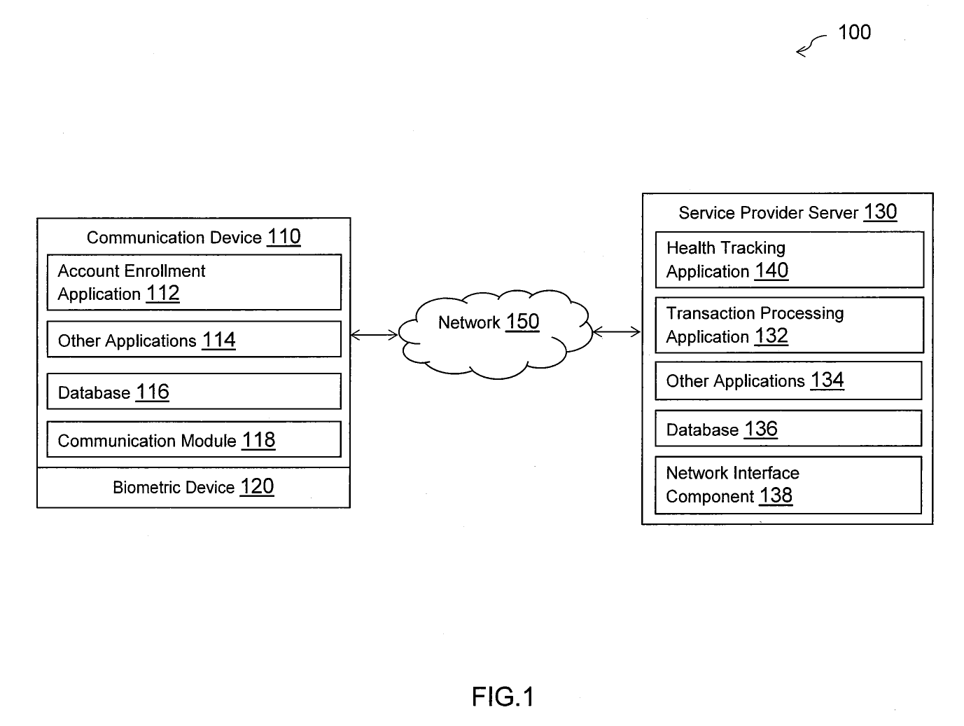

FIG. 1 is a block diagram of a networked system 100 suitable for implementing the processes described herein, according to an embodiment. As shown, system 100 may comprise or implement a plurality of devices, servers, and/or software components that operate to perform various methodologies in accordance with the described embodiments. Exemplary devices and servers may include device, stand-alone, and enterprise-class servers, operating an OS such as a MICROSOFT® OS, a UNIX® OS, a LINUX® OS, or other suitable device and/or server based OS. It can be appreciated that the devices and/or servers illustrated in FIG. 1 may be deployed in other ways and that the operations performed and/or the services provided by such devices and/or servers may be combined or separated for a given embodiment and may be performed by a greater number or fewer number of devices and/or servers. One or more devices and/or servers may be operated and/or maintained by the same or different entities.

System 100 includes a communication device 110, a biometric device 120, and a service provider server 130 in communication over a network 150. A user (not shown) may utilize communication device 110 to utilize the various features available for communication device 110, which may include processes and/or applications associated with biometric device 120 to detect and record biometric readings and activity of the user through sensed data. The user may also utilize communication device 110 to sign up and enroll in a shared account to track biometrics and provide value apportionment. In various embodiments, communication device 110 may also be used to establish the shared account. Service provider server 130 may retrieve biometric activity data detected by biometric device 120, and may utilize the data to determine an apportionment of a value for the shared account based on the biometric activity data. Communication device 110 may view output data of tracked biometric activity for the user, as well as other users, from service provider server 130, and may utilize an account of the user to electronically process transactions using the received value from service provider server 130.

Communication device 110, biometric device 120, and service provider server 130 may each include one or more processors, memories, and other appropriate components for executing instructions such as program code and/or data stored on one or more computer readable mediums to implement the various applications, data, and steps described herein. For example, such instructions may be stored in one or more computer readable media such as memories or data storage devices internal and/or external to various components of system 100, and/or accessible over network 150.

Communication device 110 may be implemented as a communication device that may utilize appropriate hardware and software configured for wired and/or wireless communication with biometric device 120 and/or service provider server 130. For example, in one embodiment, communication device 110 may be implemented as a personal computer (PC), a smart phone, laptop/tablet computer, wristwatch with appropriate computer hardware resources, eyeglasses with appropriate computer hardware (e.g. GOOGLE GLASS®), other type of wearable computing device, implantable communication devices, and/or other types of computing devices capable of transmitting and/or receiving data, such as an IPAD® from APPLE®. Although a communication device is shown, the communication device may be managed or controlled by any suitable processing device. Although only one communication device is shown, a plurality of communication devices may function similarly.

Communication device 110 of FIG. 1 contains an account enrollment application 112, other applications 114, a database 116, and a communication module 118. Account enrollment application 112 and other applications 114 may correspond to executable processes, procedures, and/or applications with associated hardware. In other embodiments, communication device 110 may include additional or different modules having specialized hardware and/or software as required.

Account enrollment application 112 may correspond to one or more processes to execute modules and associated devices of communication device 110 to establish a shared and/or personal account, as well as provide value to the shared account and link biometric device 120 to the shared account for biometric activity tracking. In this regard, account enrollment application 112 may correspond to specialized hardware and/or software utilized by communication device 110 to provide an interface to permit the user to enter input and other data for an account, including a personal or shared account. The data may be input through an input device (e.g., touch screen with a graphical user interface displayed by account enrollment application 112, keypad/keyboard, mouse, etc.) and/or through a data capture device (e.g., scanner, camera, other optical device, etc.). Information for an account may include data necessary to establish the account, including personal and/or financial data for a personal account or group data, value sharing settings and biometric goals, enrolled users, and/or biometric device identifiers for a shared account. A personal account accessible through account enrollment application 112 may be used to initiate, receive, and/or process/complete transactions using services provided by service provider server 130. A shared account may be accessible to view data, link biometric device 120, provide value for a shared value pool, or otherwise configure the shared account. Once entered, the account data may be communicated to service provider server 130 over network 150 by account enrollment application 112 for establishment of the account.

Where the account established by account enrollment application 112 corresponds to a private or personal account for a user, account enrollment application 112 may be implemented as a user interface enabling the user to enter payment instruments for use in a digital wallet that provides transaction processing services. Account enrollment application 112 may be utilized to select payment instrument(s) for use during a transaction between the user and another entity, including service provider server 130 for use in providing a part or all of a value used for a shared account and apportioned between users enrolled in the account based on detected biometric data over a time period. Account enrollment application 112 may utilize the digital wallet and/or user financial information for the personal account, such as a credit card, bank account, or other financial account, as a payment instrument when processing a transaction and/or transferring funds or value to one or more other accounts (e.g., the shared account). Account enrollment application 112 may be utilized to view the results of the transaction and/or for viewing and storage of a transaction history, such as a receipt.

Account enrollment application 112 may correspond to a general browser application configured to retrieve, present, and communicate information over the Internet (e.g., utilize resources on the World Wide Web) or a private network. For example, account enrollment application 112 may provide a web browser, which may send and receive information over network 150, including retrieving website information, presenting the website information to the user, and/or communicating information to the website, including payment information. However, in other embodiments, account enrollment application 112 may include a dedicated application of service provider server 130 or other entity (e.g., a merchant), which may be configured to assist in processing purchase requests. Moreover, in other embodiments, service provider server 130 may not perform transaction processing, and may instead correspond to another service provider, where account enrollment application 112 may include processes to access and utilize services provided by such a service provider, for example, the one or more of processes described herein.

Where a shared account is created using account enrollment application 112, the user may further use account enrollment application 112 to enter biometric activity monitoring preferences, settings, and/or goals, as well as provide and/or manage the shared account's value or pool. Input used to determine the preferences may be entered at the time of establishment of the shared account or after establishment of the account, for example, when other users enroll and/or begin sharing and tracking biometric activity data using the account. The user may set goals for value sharing settings, which may include an amount of the shared value to receive, a biometric monitoring time, and/or other data necessary to establish a value sharing setting that apportions the shared account's value based on biometric activity. The user may establish an amount (e.g., a number value) for biometrics and/or activity over the time period, or a percentage increase or decrease. The user may also establish an amount or portion of the value set for meeting or exceeding the goal, such as a flat amount or percentage amount of the shared account's value. Once the shared account is established and created, an identifier for the shared account may be provided to the user for use by other users in enrolling in the shared accounts biometric tracking services.

Once a shared account is established either by the user using communication device 110 or by another user, communication device 110 may be used to enroll in the shared account. Account enrollment application 112 may be used to access an online platform provided by service provider server 130 that includes a portal that outputs one or more interfaces of service provider server 130 through account enrollment application 112. The interfaces of the portal may include one or more fields and executable processes for the user to enter the shared account code where required, as well as provide an identifier for the user, such as an account identifier, phone number, email address, or other contact identifier allowing the user to receive and send messages with one or more users associated with the shared account. The identifier may also be used to receive value shared based on biometric activity. Account enrollment application 112 may also be used to provide an identifier for biometric device 120, which may be used by service provider server 130 to monitor biometric activity of the user through biometric device 120. The identifier may correspond to an identifier for biometric device 120, such as a device identifier. The identifier may also be for an account with a biometric activity monitoring application of other applications 114 and/or another online service provider that monitors biometric activity through biometric device 120, such as an account login name, password, or other data necessary to retrieve biometric data tracked by the application using biometric device 120. Once provided, account enrollment application 112 may then confirm enrollment of the user and biometric device 120 in the shared account.

In various embodiments, communication device 110 includes other applications 114 as may be desired in particular embodiments to provide features to communication device 110. For example, other applications 114 may include security applications for implementing client-side security features, programmatic client applications for interfacing with appropriate application programming interfaces (APIs) over network 160, or other types of applications Other applications 114 may also include email, texting, voice and IM applications that allow a user to send and receive emails, calls, texts, and other notifications through network 160. In various embodiments, other applications 114 may include financial applications, such as banking applications. Other applications 114 may also include other location detection applications, which may be used to determine a location for the user, such as a mapping, compass, and/or GPS application, which can include a specialized GPS receiver that obtains location information for communication device 110 and processes the location information to determine a location of communication device 110 and the user. Other applications may include social networking applications, media viewing, and/or merchant applications. Other applications 114 may include device interface applications and other display modules that may receive input from the user and/or output information to the user. For example, other applications 114 may contain software programs, executable by a processor, including a graphical user interface (GUI) configured to provide an interface to the user. Other applications 114 may therefore use devices of communication device 110, such as display devices capable of displaying information to users and other output devices, including speakers.

Other applications 114 may include an application that interfaces with biometric device 120 to detect biometric activity, and record the biometric activity to database 116 and/or an online service provider. Thus, one or more of other applications 114 may utilize short range wireless communications with biometric device 120 to detect and receive sensed biometric activity data, and process the data to determine biometrics and activity of a user. Other applications 114 may therefore collect, capture, and/or otherwise determine user data and other information for the user. The users actions may correspond to biometrics, which may be captured with the assistance of a connected device, such as a pedometer, heart rate sensor, distance tracker, etc. (e.g., a FITBIT™ or similar device using a short range wireless communication with communication device 110).

Communication device 110 may further include database 116 stored to a transitory and/or non-transitory memory of communication device 110, which may store various applications and data and be utilized during execution of various modules of communication device 110. Thus, database 116 may include, for example, identifiers such as operating system registry entries, cookies associated with account enrollment application 112 and/or other applications 114, identifiers associated with hardware of communication device 110, or other appropriate identifiers, such as identifiers used for payment/user/device authentication or identification, which may be communicated as identifying the user/communication device 110 to service provider server 130. Database 116 may include data for an account, including an account token, and associated information used by account enrollment application 112, as well as data determined using other applications 114, such as user biometrics and activity information, which may be communicated to service provider server 130.

Communication device 110 includes at least one communication module 118 adapted to communicate with biometric device 120 and/or service provider server 130. In various embodiments, communication module 118 may include a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency, infrared, Bluetooth, and near field communication devices. Communication module 118 may communicate directly with nearby devices using short range communications, such as Bluetooth Low Energy, LTE Direct, WiFi, radio frequency, infrared, Bluetooth, and near field communications.

Biometric device 120 may correspond to a device associated with communication device 110 and used by the user of communication device 110, which may utilize appropriate hardware and software configured for wired and/or wireless communication with at least one of communication device 110 and/or service provider server 130. For example, biometric device 120 may be communicatively coupled to communication device 110 through short range wireless communications. Biometric device 120 may include short range wireless communication components, which may utilize short range wireless communications to communication with communication device 110 (e.g., over Bluetooth Low Energy, LTE Direct, WiFi, radio frequency, infrared, Bluetooth, near field communications, etc.). In other embodiments, biometric device 120 may further include network communications and be capable of transmitting and/or receiving information from service provider server 130. Biometric device 120 may include a sensor or other component used to collect the information associated with the user, such as an input device, a camera, a microphone, an accelerometer, a motion detector, an environmental sensor, and/or a biometric sensor. The current information may correspond to biometric activity data, which may correspond to biometrics of the user (e.g., heart rate, perspiration, etc.) and/or activity (e.g., steps taken, distance traveled, exercise performed, etc.). Biometric device 120 may store the biometric activity data, or may transmit the biometric activity data to communication device 110 and/or service provider server 130 using the communication component. Biometric device 120 may include display devices, including GUI's capable of displaying information to users. Biometric device 120 may also include other output devices, including speakers. Biometric device 120 may include input devices, including touch screens. In various embodiments, biometric device 120 may correspond to a wristwatch with appropriate computer hardware resources, eyeglasses with appropriate computer hardware (e.g. GOOGLE GLASS™), other type of wearable computing device, implantable communication devices, and/or other types of computing devices capable of transmitting and/or receiving data, such as a FITBIT™.

Service provider server 130 may be maintained, for example, by an online payment service provider, which may provide account services and/or processing for financial transactions on behalf of users. In this regard, service provider server 130 includes one or more processing applications which may be configured to interact with communication device 110, biometric device 120, and/or another device/server to facilitate establishment of a shared account between users and tracking of biometric data. In one example, service provider server 130 may be provided by PAYPAL®, Inc. of San Jose, Calif., USA. However, in other embodiments, service provider server 130 may be maintained by or include a credit provider, financial services provider, financial data provider, and/or other service provider, including mobile application service providers, social networking and/or microblogging platforms, messaging services, or other online resources that may be utilized by users to establish personal accounts and use the personal accounts to engage is value apportioning of a shared account using biometric data. For example, service provider server 130 may correspond to a social networking or messaging platform allowing users having personal accounts to enroll in a shared account and link biometric devices for the purpose of value apportionment.

Service provider server 130 of FIG. 1 includes a health tracking application 140, a transaction processing application 132, other applications 134, a database 136, and a network interface component 148. Health tracking application 140, transaction processing application 132, and other applications 134 may correspond to executable processes, procedures, and/or applications with associated hardware. In other embodiments, service provider server 130 may include additional or different modules having specialized hardware and/or software as required.

Health tracking application 140 may correspond to one or more processes to execute modules and associated specialized hardware of service provider server 130 to access shared account data for a shared account having one or more enrolled users and a value that is apportioned based on biometric activity data detected over time, and apportion the value based on the data. In this regard, health tracking application 140 may correspond to specialized hardware and/or software to first access value sharing goals, settings, and/or preferences using shared account. The value sharing settings for the shared account may include information used to determine when a goal for value sharing of the shared account's value is met using biometric activity data and/or an amount or share of the account's value earned based on whether the goal was met. Health tracking application 140 may further request biometric activity data and/or information identifying biometric device 120, including an identifier, for use in retrieving biometric activity data. Health tracking application 140 may further receive, access, and/or determine the biometric activity data for the user, which may include a location or travel data (e.g., through a GPS transceiver or other location determining system), biometrics, activity, and/or other data associated with health or fitness of the user.

Using the settings for the account and the biometric activity data, health tracking application 140 may determine a share or portion of the shared account's value earned by the user based on goals or settings met, achieved, or exceeded through the user's biometric activity data. The share or portion may correspond to a pro rata share based on the biometric activity data, which may therefore be proportional to the user's biometric activity data as compared to the setting, other users, and/or amount exceeding the setting or a previous biometric activity marker of the user. Health tracking application 140 may utilize transaction processing application 132 to provide the share to a personal account of the user, which may be utilized by communication device 110. Additionally, health tracking application 140 may provide output data for one or more interfaces that includes biometric activity data for the user, as well as comparison biometric activity data for one or more other enrolled users in the shared account. For example, health tracking application 140 may provide static or real-time data output tracking the user's biometrics and/or activity, which may also be compared to corresponding data of other users. Health tracking application 140 may hide, redact, or otherwise obscure identifiers for other users associated with the other biometric activity data in certain embodiments, which may be according to preferences or settings to provide privacy to users. Additionally, health tracking application 140 may track the user's biometric activity data over one or more periods of time, which may be output as increases and/or decreases in the data over the time period(s). Moreover, the data output through the interfaces may show earned goals or matched/exceeded biometric activity settings, as well as necessary biometrics or activity to meet pending goals.

Transaction processing application 132 may correspond to one or more processes to execute software modules and associated specialized hardware of transaction processor server 140 to provide account and transaction processing services to users, for example though an account and/or payment instruments of the user, where the account services may be utilized to establish a shared account. In this regard, transaction processing application 132 may correspond to specialized hardware and/or software to provide an online shared account generation and maintenance platform, where a user associated with communication device 110 may provide the aforementioned account information. The user may provide necessary data for the shared account through communication device 110, which may include an account name, initial seed value or amount for the shared account, goals or settings, and/or users requested to join the shared account for biometric activity tracking. Transaction processing application 132 may establish the account using the provided data, and may provide a code or identifier for the account that allows signup and biometric monitoring device linking to other users enrolling in the shared account.

In order to establish a personal account to send and receive payments, transaction processing application 132 may receive information requesting establishment of the account. The information may include user personal, business, and/or financial information, or other information for an entity establishing the account (e.g., personal user, merchant, charity, etc.). Additionally the information may include a login, account name, password, PIN, or other account creation information. The user establishing the account may provide a name, address, social security number, or other personal or business information necessary to establish the account and/or effectuate payments through the account. Transaction processing application 132 may further allow the entity to service and maintain the payment account, for example, by adding and removing funding instruments. Once the shared account and/or personal account is established, transaction processing application 132 may process a transaction to transfer a value from a personal account to the shared account. Health tracking application 140 may be used to track biometric activity and, based on the biometric activity of a user, determine a share or portion of a value for the shared account to provide each user based on their biometric activity. Transaction processing application 132 may be used to process a transaction to withdraw the amount of the share of the value from the shared account and deposit the amount to the personal account. Additionally, transaction processing application 132 may establish one or more restrictions on use of the amount, which may be enforced during electronic transaction processing by transaction processing application 132.

In various embodiments, service provider server 130 includes other applications 134 as may be desired in particular embodiments to provide features to payment provider server 134. For example, other applications 134 may include security applications for implementing server-side security features, programmatic client applications for interfacing with appropriate application programming interfaces (APIs) over network 150, or other types of applications. Other applications 134 may contain software programs, executable by a processor, including a graphical user interface (GUI), configured to provide an interface to the user when accessing payment provider server 134, where the user or other users may interact with the GUI to more easily view and communicate information. In various embodiments, other applications 134 may include connection and/or communication applications, which may be utilized to communicate information to over network 150.

Additionally, service provider server 130 includes database 136. As previously discussed, the user may establish one or more payment accounts having a digital wallet with service provider server 130. Accounts in database 136 may include user/merchant information, such as name, address, birthdate, payment instruments/funding sources, additional user financial information, user preferences, and/or other desired user data. The user and/or the merchant may link to their respective digital wallets and/or payment accounts through an account, user, merchant, and/or device identifier. Thus, when an identifier is transmitted to service provider server 130, e.g. from communication device 110, one or more digital wallets and/or payment accounts belonging to the user and/or the merchant may be found. Database 136 may also store the shared account data for the shared account enrolled in by one or more users. Database 136 may also include biometric activity data transmitted to service provider server 130 from one or more biometric activity monitoring devices, accounts, and/or service provides, as well as output data processed and determined using the biometric activity data and information for earned shares of a value based on biometric activity.

In various embodiments, service provider server 130 includes at least one network interface component 148 adapted to communicate communication device 110 and/or biometric device 120 over network 150. In various embodiments, network interface component 148 may comprise a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency (RF), and infrared (IR) communication devices.

Network 150 may be implemented as a single network or a combination of multiple networks. For example, in various embodiments, network 150 may include the Internet or one or more intranets, landline networks, wireless networks, and/or other appropriate types of networks. Thus, network 150 may correspond to small scale communication networks, such as a private or local area network, or a larger scale network, such as a wide area network or the Internet, accessible by the various components of system 100.

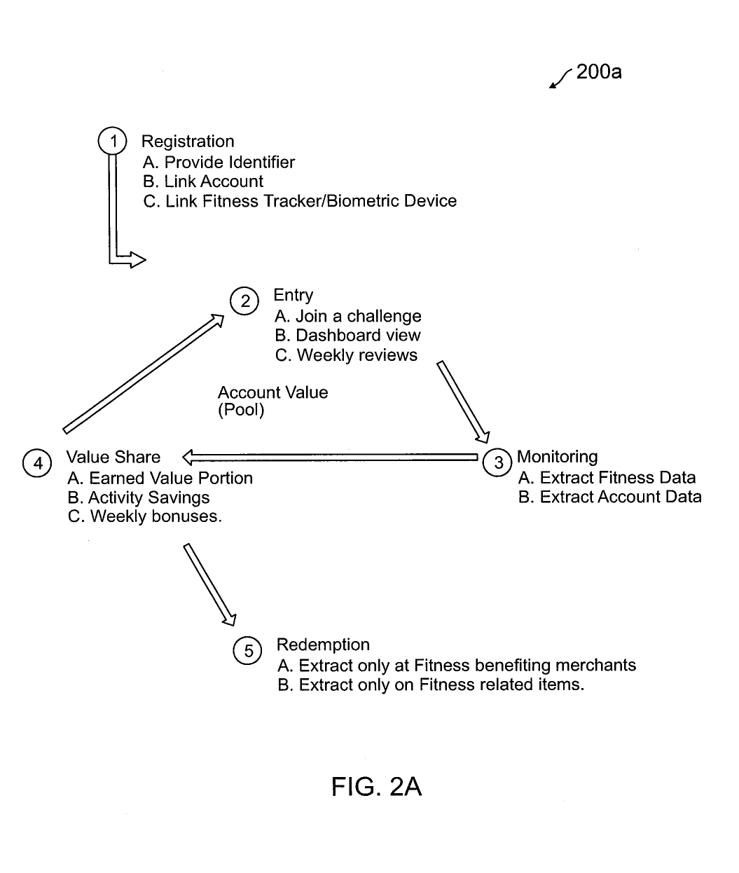

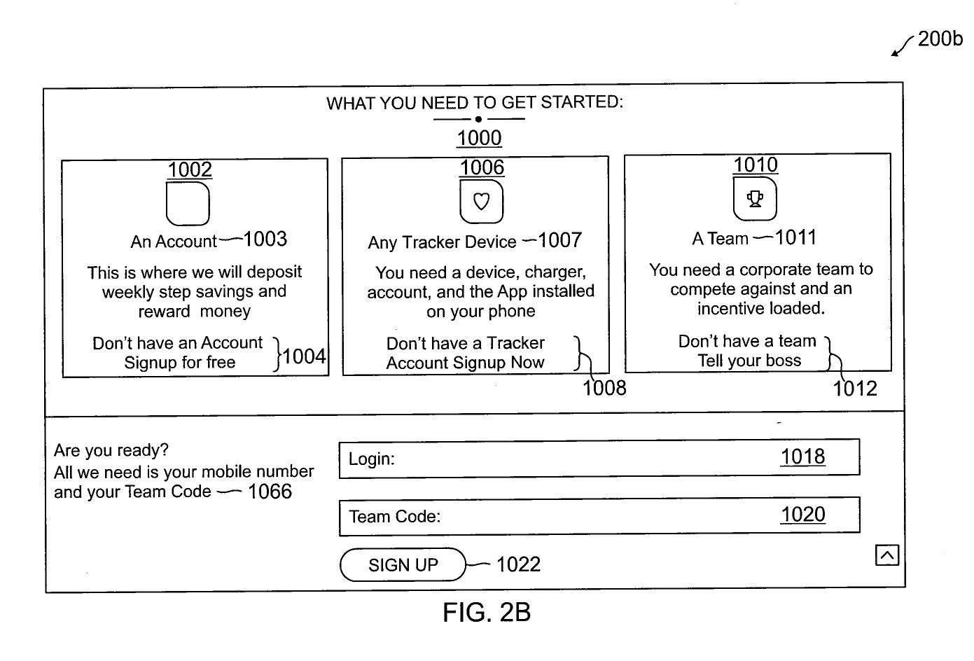

FIG. 2A is an exemplary interaction flowchart for registration biometric devices in a shared value account and tracking of biometric activity data for value apportionment, according to an embodiment. Note that one or more steps, processes, and methods described herein may be omitted, performed in a different sequence, or combined as desired or appropriate.

Flowchart 200a of FIG. 2A includes a process for an enrolling user to enroll in a shared account and perform biometric activity monitoring. Flowchart 200a begins at phase 1 for registration. Phase 1 may be provided through a portal of a biometric tracking platform provided by an online service provider that provides one or more processes and interfaces to input data for account signup and enrollment in the shared account. During registration of phase 1, an enrolling user may be required by the service provider's platform to provide an identifier at step 1A of phase 1, which may correspond to a shared account identifier used to identify the shared account the user wishes to enroll in. The user may then link an account at step 1B of phase 1, for example, a personal account, which may be linked to the shared account to allow for transfers between the account, including depositing, by the user using the personal account, funds into the shared account to establish a value or pool for the shared account, and withdrawing from the value or pool based on the user's biometric activity. The user may then be required to link a fitness tracker or biometric device at step 1C of phase 1. The tracker or device may be linked by providing a tracker/device-specific identifier, or through linking an account with an application or online service provider that communicates with the tracker/device to track/detect biometric activity data. Once completed with phase 1, the user may move to phase 2, during which the user may perform entry processes in the shared account. During phase 2, the platform portal may allow the user to make selections and view interfaces and output data. For example, at step 2A, the user may join one or more challenges, which may have goals of required biometrics or activity to be met to receive a reward, such as all or a portion of a value for the shared account. At step 2B, the user may view a dashboard of available challenges, as well as tracked biometric data for the user and other users. For example, at step 2C, the user may view weekly reviews of past biometric activity data over the week, as well as earned and unearned rewards from challenges joined in step 2A.

During phase 3, the service provider may execute one or more processes of the biometric tracking platform to perform monitoring processes of biometric data. The service provider may extract fitness data, such as biometrics and/or activity, for a user over a time period set for a joined challenge, and may compare the data to the joined challenges, as well as any other user's fitness data, to determine if the user has won, beat, or accomplished a challenge. At step 3B, the service provider may also extract account data for the enrolled accounts, which may include user data and parameters (e.g., personal user information), as well as financial and transfer data necessary to effectuate a transfer of value or funds in a pool earned by the user through the user's fitness data and the joined challenges. If the user has accomplished or won a joined challenge, the service provider's platform may continue to phase 4, where the service provider may provide value sharing. For example, at step 2A, the service provider may provide the earned value portion of the account value to the user through processing a transfer to the personal account of the user. The service provider's platform may also display an activity savings, such as an amount earned or saved from being added to the account value due to the user's biometric activity data. At step 3C, any weekly bonuses based on user/account-specific activities may also be processed to the user's account. The cycle may then repeat to phase 2 after the time period to enable the user to repeat a challenge and/or join new challenges. The platform may also proceed to phase 5 for certain value payouts or transfers, where redemption of the transferred value is requested by the user. At step 5A, the platform may impose a limitation where the value may only be extracted at fitness benefiting merchants. Similarly, the platform may only allow extraction and redemption of the value for fitness related items at step 5B.

FIG. 2B is an exemplary application interface to register and enroll in a shared account for biometric activity data, according to an embodiment. Environment 200b of FIG. 2B includes an interface 1000 that may be output when a shared account signup and enrollment portal of a biometric tracking platform, which may be accessed by a device, such as communication device 110 discussed in reference to system 100 of FIG. 1. The platform's portal may be provided by and accessed from an online service provider, such service provider server 130 discussed in reference to system 100 of FIG. 1. Thus, interface 1000 may be displayed by communication device 110 of system 100.

In environment 200, interface 1000 includes one or more interface elements or fields that may be interacted with by a user to perform account enrollment in a shared account. In this regard, interface 1000 first includes an account linking option 1002, where account linking option 1002 may be used to provide information to link a personal account to a shared account. For example, text 1003 may provide a user with information necessary to link an account, such as a required identifier and/or account type. If the user does not have an account required in text 1003, personal account signup 1004 may be used to establish a required personal account for the user.

A device linking option 1006 may also be used to provide information to link a biometric activity tracking device to a shared account that may be enrolled in by a user through interface 1000. Text 1007 may provide the user with information necessary to determine a device type and/or device identifier for a device that tracks a user's biometrics and activity, and what devices may be linked to the shared account. If the user does not have a device or cannot find the correct information to link the device, such as a device identifier or a biometric activity tracking account used with the device, device option 1008 may be selected to guide a user through a process to retrieve or determine such information and/or receive offers for such devices (e.g., through a signup process for a tracker device/account).

A team signup option 1010 may also be used to provide information necessary for a user to locate a shared account and signup or enroll in the shared account. Text 1011 may display the information necessary to identify and enroll in a shared account, such as an account code or other account identifier. Where a user does not know or have an account code or identifier, a shared account signup 1012 may provide instructions for a user on how to find or determine a code, and may also provide a process to establish a shared account if no shared account exists that is available for the user to join. In order to initiate the process, a signup field 1014 may be displayed, which may display a message 1016 instructing a user of required information to be entered to the fields of interface 1000. A login field 1018 may provide an input field for a user to enter a login for a personal account or identifier that may be used to identify the user and retrieve the user's information, as well as send and receive funds or value. The identifier placed in login field 1018 may be used to provide value to the shared account, such as value placed into a pool available for rewards and/or payout based on biometric activity. The identifier may also be used to transfer the rewards and/or payout from the pool to the person account of the user. Interface 1000 also includes a team code field to provide the team code to identify the account. Once entered, the user may select signup option 1022 to finish enrollment in the shared account. Additionally, on one or more additional interfaces, the user may link their device using the processes described in device linking option 1006.

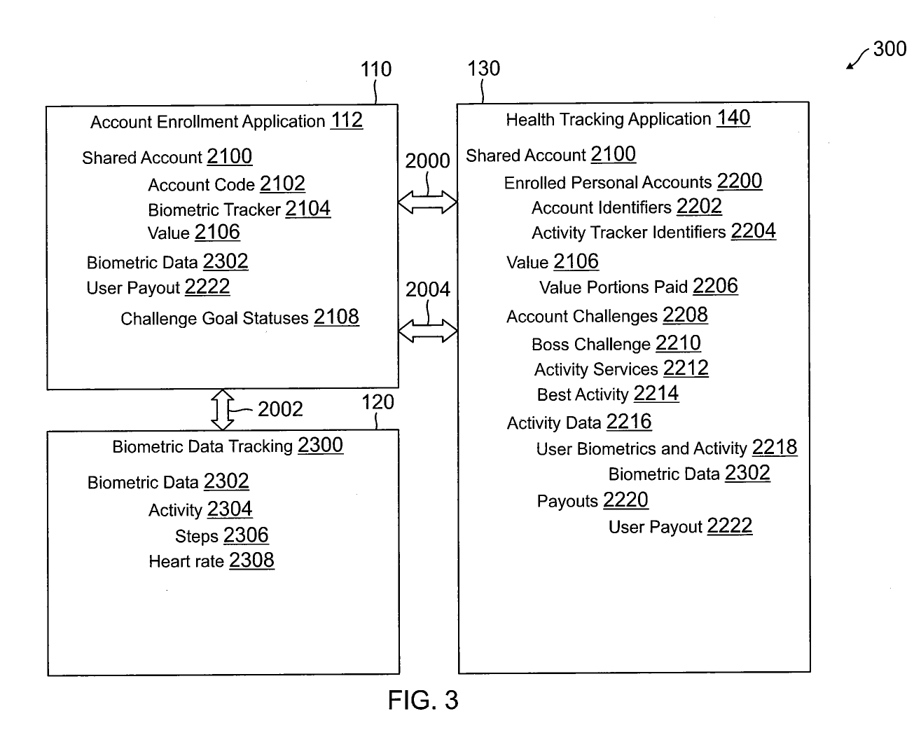

FIG. 3 is an exemplary system environment where a biometric tracking portal may interface with a communication device and biometric tracking device to determine value apportionment based on biometric activity data, according to an embodiment. Environment 300 of FIG. 3 includes account enrollment application 112 and health tracking application 140, which may be executed by communication device 110 and service provider server 130, respectively, discussed in reference to system 100 of FIG. 1. Moreover, FIG. 3 includes a biometric device 120 discussed in reference to system 100 of FIG. 1.

Communication device 110 executes account enrollment application 112 corresponding generally to the specialized hardware and/or software modules and processes described in reference to FIG. 1. Account enrollment application 112 may be used to access and enroll in a shared account 2100. Shared account 2100 may be accessed and enrolled in using account code 2102. After submitting account code 2102, biometric tracker 2104 may be linked to shared account 2100, and a value 2106 may be provided to shared account 2100. Such data may be exchanged with health tracking application 140 of service provider server 130 over communication exchange 2000.

Service provider server 130 may execute health tracking application 140 corresponding generally to the specialized hardware and/or software modules and processes described in reference to FIG. 1. In this regard, health tracking application 140 includes data for shared account 2100, which may include enrolled personal accounts 2200. Personal accounts 2200 may be identifier through account identifiers 2202 and may be associated with activity tracker identifier 2204 for devices that may monitor biometrics and health for users associated with personal accounts 2200. Additionally, shared account 2100 may include value 2106 and account challenges 2208, including a boss challenge 2210 where employees may attempt to outpace or beat activity performed by a boss daily or weekly. Account challenges 2208 may include activity savings 2212, which may correspond to payouts, rewards, and savings earned based on the biometric activity of users. Additionally, a best activity 2214 may reward a user that achieves a highest amount, number, or percentage increase in biometric activity over a time period.

In order to provide value portions paid 2206 for value 2106, health tracking application 140 may require biometric data 2302 from biometric device 120. Health tracking application 140 may query account enrollment application 112 for biometric data 2302, which may be retrieve from a process for biometric data tracking 2300 of biometric device 120. Biometric data 2302 detected by biometric data tracking 2300 may include activity 2304, such as steps 2306 taken by a user using or wearing biometric device 120, as well as heart rate 2308 detected for the user. Account enrollment application 112 may receive biometric data 2302 from biometric device 120 over communication exchange 2002. Communication exchange 2002 may be continuous so that biometric data 2302 is continuously loaded to communication device 110, or may be retrieved based on a query by health tracking application 140 of service provider server 130. After retrieval, communication exchange 2004 may be performed by communication device 110 and service provider server 140 to provide biometric data to health tracking application 140. Health tracking application may then process activity data 2216 for all users, which includes user biometrics and activity (e.g., biometric data 2302 from biometric device 120). Health tracking application 140 may then determine payouts 2220 having a user payout 2222. Communication exchange 2004 may be used to provide user payout 2222 to account enrollment application 112, which may display challenge goal statuses 2108 that provided user payouts 2222.

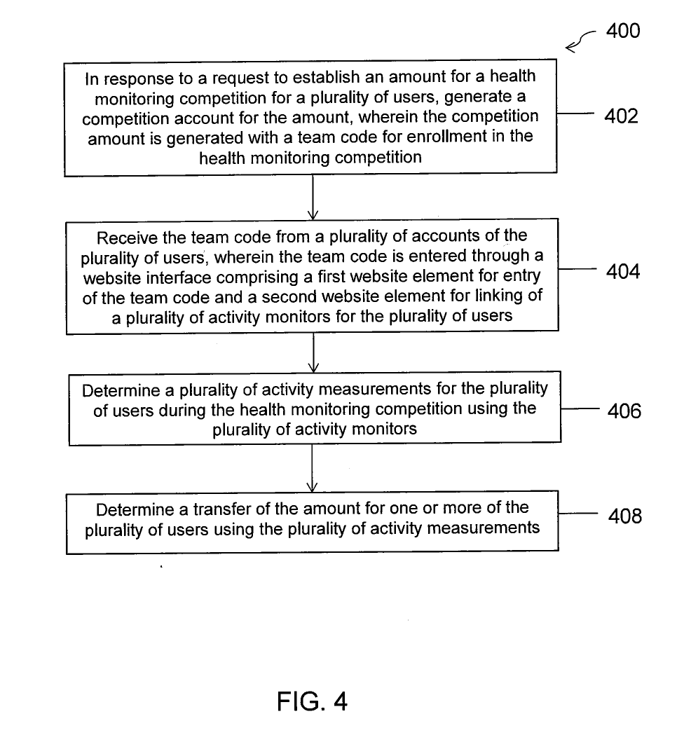

FIG. 4 is a flowchart of an exemplary process for shared account enrollment and biometric activity monitoring for value apportionment, according to an embodiment. Note that one or more steps, processes, and methods described herein may be omitted, performed in a different sequence, or combined as desired or appropriate.

At step 402 of flowchart 400, in response to a request to establish an amount for a health monitoring competition for a plurality of users, a competition account for the amount is generated, wherein the competition account is generated with a team code for enrollment in the health monitoring competition. This may include requesting a portion of the amount from accounts for the plurality of users, wherein the portion is evenly distributed over the accounts and in response to receiving the portion from each of the accounts, generating the competition account with the team code. The amount may also be received from an account of a user unaffiliated with the pool that is outside of the plurality of users, or that is a creator of the amount and the account and is one of the plurality of users. The amount may then be established as a pool for the competition account. The team code is received from a plurality of accounts of the plurality of users, wherein the team code is entered through a web site interface comprising a first web site element for entry of the team code and a second website element for linking of a plurality of activity monitors for the plurality of users, at step 404.

At step 406, a plurality of activity measurements for the plurality of users during the health monitoring competition is determined using the plurality of activity monitors. The activity measurements may include a tracked exercise amount, a heart rate, a number of daily footsteps taken, or an amount of weight loss. The activity monitors may therefore be associated with or contained within a wearable or mobile computing device, such as a heart rate sensor, a pedometer, a distance tracker, or an activity tracker. In order to track the plurality of measurements, the team code may be received from each of the plurality of accounts through a signup interface of an account enrollment process, and an interface comprising an interactive process to link each of the plurality of activity monitors may be output. In response to receiving an identifier for the each of the plurality of activity monitors, they may be linked to the account. One or more of the activity monitors may be linked to the account through an online platform that tracks biometric activity data, such as the activity measurements. An activity monitor may also be connected to a computing device that uploads the biometric activity data to the online platform.

A transfer of the amount for one or more of the plurality of users is determined using the plurality of activity measurements, at step 408. The transfer may instead correspond to at least a portion of the amount, and may correspond to a pro rata share. This may further be based on value sharing or activity reward settings. This may include apportioning the amount over the plurality of users based on the plurality of activity measurements. For example, the amount may be apportioned based on an increase or a decrease in the plurality of activity measurements over a time period that meets a goal set for the account. The amount may also be apportioned to another user, such as a second user based on the second user's activity measurements. The amount may be apportioned based on a comparison of each of the plurality of users and their activity measurements. Additional activity measurements or other biometric data may be received, for example, over another time period. Thus, additional transfers may be processed from the amount based on the activity measurements during the subsequent time period.

In order to process the transfer, a transaction between the competition account and one or more personal account of the plurality of users may be processed by deducting the amount from the competition amount and providing to the personal account(s). The amount may comprise a monetary amount provided in the competition account from an account administrator. The account administrator may require that the amount provided to the personal account(s) is only valid for specific merchants established by the account administrator. An interface may also be provided that comprises the activity measurements, such as biometric activity data, which may display the portion of the amount earned by each of the users.

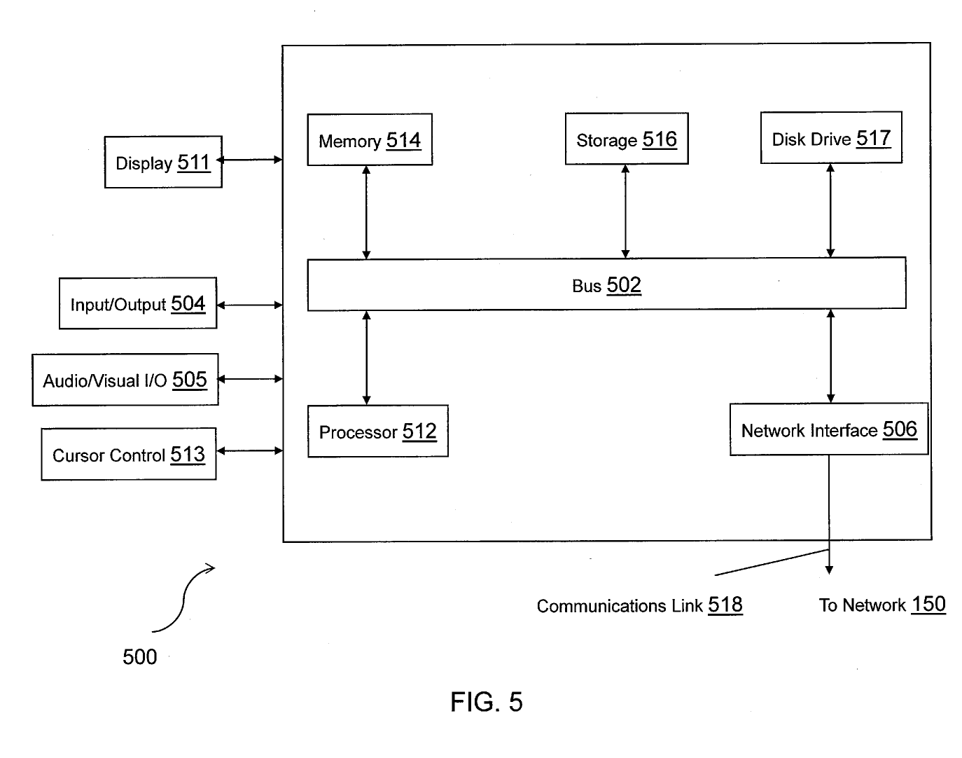

FIG. 5 is a block diagram of a computer system suitable for implementing one or more components in FIG. 1, according to an embodiment. In various embodiments, the communication device may comprise a personal computing device (e.g., smart phone, a computing tablet, a personal computer, laptop, a wearable computing device such as glasses or a watch, Bluetooth device, key FOB, badge, etc.) capable of communicating with the network. The service provider may utilize a network computing device (e.g., a network server) capable of communicating with the network. It should be appreciated that each of the devices utilized by users and service providers may be implemented as computer system 500 in a manner as follows.

Computer system 500 includes a bus 502 or other communication mechanism for communicating information data, signals, and information between various components of computer system 500. Components include an input/output (I/O) component 504 that processes a user action, such as selecting keys from a keypad/keyboard, selecting one or more buttons, image, or links, and/or moving one or more images, etc., and sends a corresponding signal to bus 502. I/O component 504 may also include an output component, such as a display 511 and a cursor control 513 (such as a keyboard, keypad, mouse, etc.). An optional audio input/output component 505 may also be included to allow a user to use voice for inputting information by converting audio signals. Audio I/O component 505 may allow the user to hear audio. A transceiver or network interface 506 transmits and receives signals between computer system 500 and other devices, such as another communication device, service device, or a service provider server via network 150. In one embodiment, the transmission is wireless, although other transmission mediums and methods may also be suitable. One or more processors 512, which can be a micro-controller, digital signal processor (DSP), or other processing component, processes these various signals, such as for display on computer system 500 or transmission to other devices via a communication link 518. Processor(s) 512 may also control transmission of information, such as cookies or IP addresses, to other devices.

Components of computer system 500 also include a system memory component 514 (e.g., RAM), a static storage component 516 (e.g., ROM), and/or a disk drive 517. Computer system 500 performs specific operations by processor(s) 512 and other components by executing one or more sequences of instructions contained in system memory component 514. Logic may be encoded in a computer readable medium, which may refer to any medium that participates in providing instructions to processor(s) 512 for execution. Such a medium may take many forms, including but not limited to, non-volatile media, volatile media, and transmission media. In various embodiments, non-volatile media includes optical or magnetic disks, volatile media includes dynamic memory, such as system memory component 514, and transmission media includes coaxial cables, copper wire, and fiber optics, including wires that comprise bus 502. In one embodiment, the logic is encoded in non-transitory computer readable medium. In one example, transmission media may take the form of acoustic or light waves, such as those generated during radio wave, optical, and infrared data communications.

Some common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EEPROM, FLASH-EEPROM, any other memory chip or cartridge, or any other medium from which a computer is adapted to read.

In various embodiments of the present disclosure, execution of instruction sequences to practice the present disclosure may be performed by computer system 500. In various other embodiments of the present disclosure, a plurality of computer systems 500 coupled by communication link 518 to the network (e.g., such as a LAN, WLAN, PTSN, and/or various other wired or wireless networks, including telecommunications, mobile, and cellular phone networks) may perform instruction sequences to practice the present disclosure in coordination with one another.

Where applicable, various embodiments provided by the present disclosure may be implemented using hardware, software, or combinations of hardware and software. Also, where applicable, the various hardware components and/or software components set forth herein may be combined into composite components comprising software, hardware, and/or both without departing from the spirit of the present disclosure. Where applicable, the various hardware components and/or software components set forth herein may be separated into sub-components comprising software, hardware, or both without departing from the scope of the present disclosure. In addition, where applicable, it is contemplated that software components may be implemented as hardware components and vice-versa.

Software, in accordance with the present disclosure, such as program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein.

The foregoing disclosure is not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, persons of ordinary skill in the art will recognize that changes may be made in form and detail without departing from the scope of the present disclosure. Thus, the present disclosure is limited only by the claims.

What is claimed is:

1. A system comprising:
a shared account signup platform comprising a biometric tracking portal;
a network interface component comprising:
an input component configured to a request to access the biometric tracking portal on the shared account signup platform; and
an output component configured to output the biometric tracking portal;
a non-transitory memory storing instructions; and
one or more hardware processors couple to the non-transitory memory and configured to read the instructions from the non-transitory memory to cause the system to perform operations comprising:
receiving, from a first device of a first user, an indication of the first user logging into the biometric tracking portal through a first account of the first user;
in response to the indication, receiving an account identifier for a shared account from a shared account enrollment interface of the biometric tracking portal;
receiving a device identifier from the first device;
linking the first device to the shared account;
determining, through the first device, first biometric activity data for the first user over a first time period set for the shared account;
in response to an expiration of the first time period, determining value sharing settings for the shared account based on the first biometric activity data;
determining a first pro rata share of a first value associated with the shared account based on the first biometric activity data and the value sharing settings; and
providing the first pro rata share of the first value from the shared account to the first account.

2. The system of claim 1, wherein prior to the determining the first pro rata share of the first value, the operations further comprise:
determining second biometric activity data for the first user over a second time period occurring prior to the first time period; and
determining a first percentage increase or a first percentage decrease over the first time period and the second time period for the first biometric activity data and the second biometric activity data,
wherein the first pro rata share is further determined based on the first percentage increase or the first percentage decrease.

3. The system of claim 2, wherein prior to the determining the first pro rata share of the first value, the operations further comprise:
determining, through a second device link to the shared account, third biometric activity data for a second user over the first time period and the second time period;
determining a second percentage increase or a second percentage decrease over the first time period and the second time period for the third biometric activity data;
comparing the first percentage increase or the first percentage decrease to the second percentage increase or the second percentage decrease; and
determining a second pro rata share of the first value for one of the first user and the second user based on the comparing.

4. The system of claim 1, wherein the first device is linked to the shared account through an online platform that tracks the first biometric activity data for the first user through the first device, and wherein first device is connected to a computing device that uploads the first biometric activity data to the online platform.

5. The system of claim 1, wherein the operations further comprise:
determining second biometric activity data for each of a plurality of users including the first user;
wherein the first pro rata share is further determined based on the second biometric activity data for each of the plurality of users.

6. The system of claim 5, wherein the operations further comprise:
determining a plurality of pro rata shares for each of the plurality of users over the first time period based on the second biometric activity data for each of the plurality of users.

7. The system of claim 6, wherein prior to the determining the first biometric activity data, the operations further comprise:
receiving a plurality of second values from each of the plurality of users, wherein each of the plurality of second values comprise a portion of the value to be established for the shared account; and
generating the value of the shared account using the plurality of second values.

8. The system of claim 6, wherein prior to the determining the first biometric activity data, the operations further comprise:
receiving the first value from a second account of a second user, wherein the second account comprises a creator of the shared account for the plurality of users, wherein the determining the first pro rata share of the first value is further based on comparing the first biometric activity data to third biometric activity data for the creator of the shared account.

9. The system of claim 1, wherein the providing the first pro rata share comprises:
processing a transaction between the first account and the shared account, wherein the transaction deducts an amount of the first pro rata share from the shared account and credits the first pro rata share to the first account.

10. The system of claim 1, wherein the first device comprises one of a wearable computing device or a mobile computing device, and wherein the first device further comprises at least one of a heart rate sensor, a pedometer, a distance tracker, or an activity tracker.

11. The system of claim 1, wherein the operations further comprise:
outputting an interface comprising the first biometric activity data with the value of the shared account.

12. The system of claim 11, wherein the interface displays an amount of the value able to be earned over the first time period based on activity of enrolled users in an activity monitoring program associated with the shared account

13. The system of claim 1, wherein the first value comprises a monetary amount provided in the shared account from an account administrator of the shared account, and wherein the first pro rata share of the first value is only valid for specific merchants established by the account administrator.

14. The system of claim 13, wherein the operations further comprise:
receiving a transaction processing request using the first pro rata share;
determining a merchant associated with the transaction processing request meets the specific merchants established for the shared account; and
processing the transaction processing request based on the merchant and the specific merchants.

15. A method comprising:
determining first motion data for a plurality of users during a time period using a plurality of activity monitoring devices associated with the plurality of users;
determining a plurality of personal accounts for the plurality of users using an identifier for each of the plurality of activity monitoring devices linked to each of the plurality of personal accounts;
determining a team account enrolled in by each of the plurality of personal accounts using an account code shared between each of the plurality of personal accounts;
determining a pool available for the team account and an activity reward setting for the pool based on the first motion data for the plurality of users; and
apportioning the pool between the plurality of users based on the activity reward setting and the first motion data.

16. The method of claim 15, wherein prior to determining the first motion data, the method further comprises:
receiving the account code from one of the plurality of personal accounts through a signup interface of an account enrollment process for the account code;
outputting an interface comprising an interactive process to link one of the plurality of activity monitoring devices; and
in response to receiving an identifier for the one of the plurality of activity monitoring devices, linking the one of the plurality of activity monitoring devices to the team account.

17. The method of claim 15, wherein prior to determining the first motion data, the method further comprises:
requesting a portion of the pool from each of the plurality of personal accounts, wherein the portion is evenly distributed over the plurality of personal accounts;
in response to receiving the portion from each of the plurality of person accounts, generating the team account with the account code; and
establishing the pool for the team account.

18. The method of claim 15, wherein the pool is provided by a first user separate from the plurality of users, and wherein the method further comprises:
receiving second motion data from the first user,
wherein the pool is further apportioned based on a comparison of the first motion data for each of the plurality of users to the second motion data for the first user.

19. The method of claim 15, the first motion data includes a tracked exercise amount, a heart rate, a number of daily footsteps taken, or an amount of weight loss, and wherein the pool is apportioned based on an increase or a decrease in the first motion data over the time period meeting a goal set for the first motion data in the activity reward setting.

20. A non-transitory machine-readable medium having stored thereon machine-readable instructions executable to cause a machine to perform operations comprising:
receiving a team code from a plurality of accounts of a plurality of users for a health monitoring competition, wherein the team code is entered through a website interface comprising a first website element for entry of the team code and a second website element for linking of a plurality of activity monitors for the plurality of users;
accessing a competition account for an amount associated with the health monitoring competition for the plurality of users;
determining a plurality of activity measurements for the plurality of users during the health monitoring competition using the plurality of activity monitors; and
determining a transfer of the amount for one or more of the plurality of users based on the plurality of activity measurements and at least one goal from the competition account.
